(19) United States
(12) Patent Application Publication
(10) Pub. No.: US 2002/0049354 A1
(43) Pub. Date: Apr. 25, 2002

(54) Novel antifoulant

(75) Inventors: Jiang-Jen Lin, Taichung (TW); Jann-Chen Lin, Chiayi (TW); Wen-Hen Lo, Chiayi (TW); Wen-Shwong Hwang, Yunlin (TW); Kun-Hai Lin, Chiayi (TW)

Correspondence Address: Mr. Peter F. Corless, EDWARDS & ANGELL, LLP, 130 Water Street, Boston, MA, 02109-4280, US

(73) Assignee: Chinese Petroleum Corporation, Taipei (TW)

(21) Appl. No.: 09/797,282

(22) Filed: Mar. 2, 2001

(30) Foreign Application Priority Data
Aug. 24, 2000 (TW) 89117042

(51) Int. Cl. C07C 215/74

(52) U.S. Cl. 564/336; 564/346

(57) ABSTRACT

The present invention relates to a novel antifoulant which is prepared by the Mannich reaction of phenolic compounds, formaldehyde, and amine compounds.

The said antifoulant is soluble in oily materials and is capable of preventing fouling and inhibition of free radical polymerization. The novel antifoulants of this invention are particularly useful as antifoulants in petrochemical plants and oil refineries.

### BACKGROUND OF THE INVENTION

In a chemical plant the reactors, pipelines, heaters and heat exchangers that are generally operated under high temperatures usually form precipitated depositions, reducing pipeline passage, hindering transport of materials and transference of heat, or clogging the filters and valves, thereby reducing productivity and increasing plant costs. Therefore, in general antifoulants are used in chemical plants to prevent the occurrence of the above mentioned problems.

Among the organic precipitates, the polymers formed are the most difficult to handle. Although saturated haydrocarbons may be polymerized, generally polymers are formed by the reaction of unsaturated hydrocarbons. In general, olefins polymerize more readily than aromatics. However, aromatics can be easily made to polymerize in the presence of organometallics with hetero-atoms such as nitrogen, oxygen, and sulfur.

Antifoulants and compositions have been dislosed, e.g. in the following patent literature, U.S. Pat. Nos. 4,927,561; 5,221,461; and 5,266,186. U.S. Pat. No. 4,927,561 discloses poly-functional antifoulant compositions; U.S. Pat. No. 5,221,461 discloses antioxidant compositions and methods using catechol compounds and organic acids; and U.S. Pat. No. 5,266,186 discloses a method of using dispersants in antifouling.

In addition, U.S. Pat. No. 3,281,359 (Oberender, et al.) discloses the use of polyalkylene sulfur phosphorus compounds and alcohol and ethylene glycolates in “petroleum lubricant as cleansing-dispersing additive”. However, research indicates that some functional groups in the effective cleansing-dispersing additives for lubricants are not suitable for use in petrochemicals as antiprecipitants.

Further, U.S. Pat. No. 3,135,792 (Kluge, et. al.) discloses alkali metal salts of hydrocarbon sulfuric phosphorus acid and use of such salts in the formulation of premium motor oil. U.S. Pat. No. 4,024,051 (Shell) discloses the use of inorganic acid compounds of sulfur as antioxidants; U.S. Pat. No. 3,105,810 (Miller) discloses the use of oil-soluble alkyl-aryl sulfides as anti-precipitants; U.S. Pat. No. 4,107,030 (Slovinsky, et. al.) teaches the use of ammonium compounds of para-amino-benzenesulfonic acid as anti-precipitants.

Also, U.S. Pat. No. 4,466,905 (Butler, et. al.) discloses the use of co-inhibitors to inhibit polymerization of ethylenic aromatic compounds under heating in a process, where the co-inhibitor comprises 2,6-dinitrogen-para-cresol (DNPC) and phenylene-diamine and is suitable for distillation processes with oxygen added.

U.S. Pat. No. 3,907,745 ( Bsharah, et. al. ) discloses the use of interactive antioxidants in polymeric systems that tend to oxidize. The system comprises antioxidants (such as diamine chelates) or metallic deactivator (such as polyamine). Said antioxidants are suitable for natural rubber and synthetic rubbery polymers.

U.S. Pat. No. 4,720,566 (Martin) discloses methods and compositions for inhibition of acrylonitrile polymerization in the quencher of an acrylonitrile production line, where compositions of hydrocarbon amines and phenylenediamine are used.

U.S. Pat. No. 4,929,778 (Rohling) discloses compositions and methods for inhibition of monomer products polymerization in the manufacture, storage, and transport of ethylenic aromatic monomers. The compositions comprise phenylene diamine compounds and hindered phenolic compounds.

U.S. Pat. No. 4,061,545 (Watson) discloses the use of phenanthrene thiazine and tert-butylcatechol combined in the presence of oxygen to reduce polymerization during distillation of ethylenic aromatic compounds that polymerise easily.

U.S. Pat. No. 4,714, 750 (Grigsby Jr. et. al) discloses Mannich condensates which are useful as epoxy accelerators and curing agents.

U.S. Pat. No. 4,952,732 (Sperarza et. al ) discloses Mannich condensates of a substituted phenol and an alkylamine containing internal alkoxy groups, which condensates are useful as surfactants, corrosion inhibitors, water repellent agents, paint adhesion promoters, etc.

U.S. Pat. No. 4,006,089 (Chibnik) discloses a Mannich base products which are useful for improving the detergency properties of fuel or a lubricant.

U.S. Pat. No. 3,868,329 (Brown et. al ) discloses a grease composition containing a Mannich base. However, this patent does not mention the use of the Mannich base as an antifoulant.

U.S. Pat. Nos. 3,950,451; 3,734,965; 5,101,060; 5,120,187; and 5,098,986 disclose Mannich condensates which are useful as a curing agent for epoxy resin.

U.S. Pat. Nos. 4,913,830 and 5,387,266 disclose Manncih base condensates which are useful as a dispersant in fuels and other media.

U.S. Pat. No. 4,810,354 discloses alkoxylated Mannich product composition which deactivate metals and inhibit oxygen promoted polymerization in hydrocarbons and petrochemicals.

However, due to the presence of organic compounds such as ethylene, propylene, butadiene, styrene and other unsaturated olefins, in oil refineries and petrochemical plants the problem of fouling is severe. For this reason, the use of antifoulants in oil refineries and petrochemical plants is a necessity.

Antifoulants are specialty chemicals and are widely used in petrochemical plants and oil refineries. Currently in Taiwan antifoulants are imported to use at high temperatures of 200 to 500° C. in production and oil refining processes (including cracking, reforming and de-ethylenation steps). In the reactors hydrocarbons are oxidized and polymerized to produce fouling and deposition in pipelines and on reactor walls. Fouling over a long period of time will affect the manufacturing process and even clog valves and pipelines, hampering the conduction of heat. This causes a waste of energy, requiring shut-downs for cleaning, thereby reducing production capacity.

At this moment in Taiwan research in antifoulants is rare, and that which does exist concentrates on only formulations. Research on synthesis of new components is still in the formative state.

The inventors of the present invention facing the existing needs of antifoulants have devoted themselves to the development of novel compounds useful as antifoulants; thus they have completed the present invention.

### BRIEF DESCRIPTION OF DRAWINGS

FIG. 1 The NMR spectrum of the product obtained in Example 1.

FIG. 2 The NMR spectrum of the product obtained in Example 10.

FIG. 3 The NMR spectrum of the product obtained in Example 15.

FIG. 4 The NMR spectrum of the product obtained in Example 23.

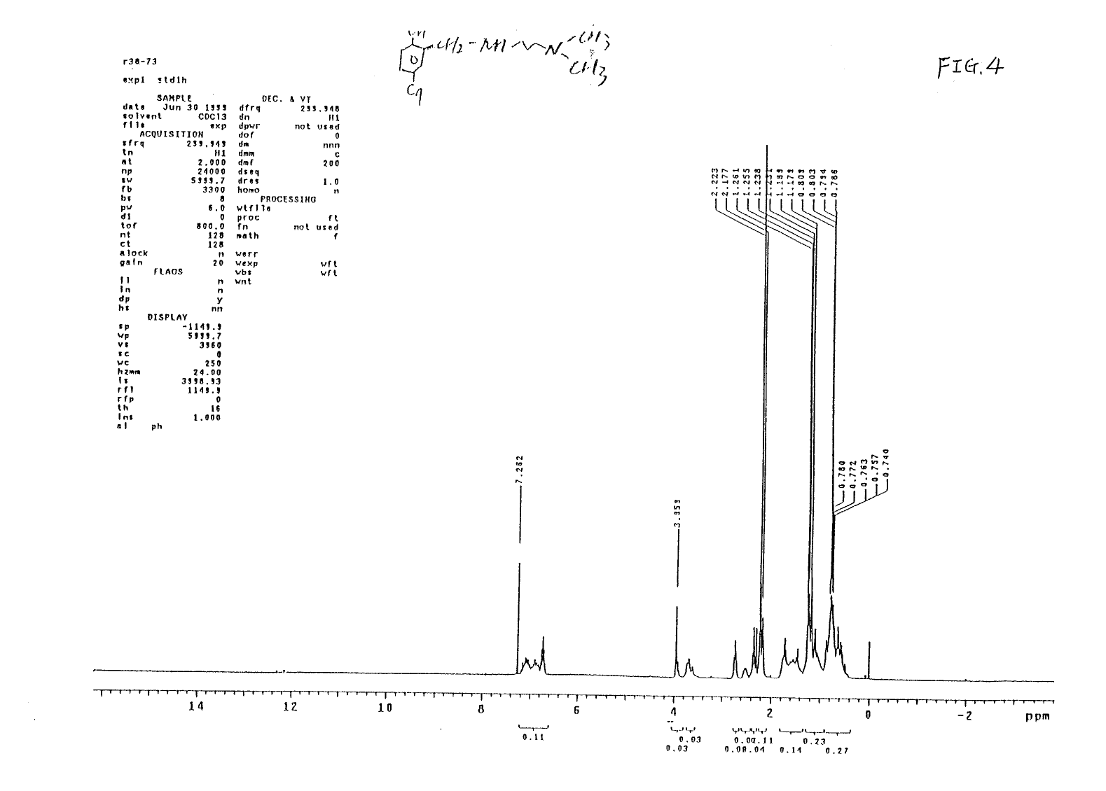

### OBJECTIVES OF THE PRESENT INVENTION

This invention relates to a novel antifoulant which is prepared by the Mannich reaction of phenolic compounds, formaldehyde, and amine compounds.

Speically, this invention relates to a novel antifoulant which is a compound having the following general formula (I):

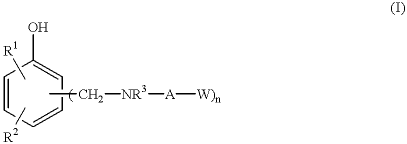

wherein R₁ and R₁ can be the same or different and independently represents hydrogen or a straight or branched chain C₁₋₁₀ alkyl group,

A represents a phenylene group which may have a C₁₋₄ alkyl substitution group,—(CH₂—NR₃—CH₂)_{z}—group (wherein z is an integer of 1 to 3), or a group of

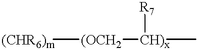

(wherein R₆ and R₇ independently represent a hydrogen atom or a straight or branched chain C₁₋₆ alkyl group, m is an integer of 1 to 6 and x represents an integer of 0 to 35),

W represents a hydroxy group or a group of the formula-NR₄R₅;

R₃ represents a hydrogen atom or a straight or branched chain C₁₋₆ alkyl group,

R₄ and R₅ can be the same or different and independently represents hydrogen atom, a straight or branched chain C₁₋₆ alkyl group, or a group of the following formula:

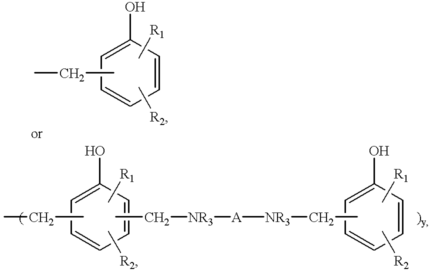

in which R₁, R₂, R₃ and A are as defined above, and y represents an integer of 1 to 3; and

n represents an integer of 1 to 3.

The antifoulant of the present invention is prepared by the Mannich reaction of phenolic compounds, formaldehyde, and amine compounds such as a compound of formula HNR₃-A-W (where R₃, A, and W are as defined above). Formaldehyde therein reacts with phenolic compounds at their meta- or para-position with respect to the hydroxy group thereof, and then reacts with the amino functional group of the amine compounds to give the compounds of the present invention, represented by the general formula (I). In other words, the group —CH₂—NR₃-A-W is attached to the meta-or para-position with respect to the hydroxyl group of the phenolic compounds.

The antifoulant of the present invention can be dissolved in oily materials, and as such it is capable of inhibiting fouling in a chemical manufacturing process. It is also capable of inhibition of free radical polymerization and is therefore a superior antifoulant.

In the compounds represented by the general formula (I) , the C₁₋₁₀ alkyl groups represented by R₁ and R₂ can be straight chain or branched alkyl groups, examples of which include, e. g., methyl, ethyl, n-propyl, iso-propyl, n-butyl, sec-butyl, tert-butyl, n-pentyl, neo-pentyl, iso-pentyl, hexyl, heptyl, octyl, nonyl, decanyl groups.

The C₁₋₄ alkyl subsitution groups of the phenylene group represented by A in the general formula (I) include methyl, ethyl, n-propyl, iso-propyl, n-butyl, sec-butyl, tert-butyl groups.

The C₁₋₆ alkyl groups represented by R₃, R4, and R₅ in the general formula (I) include methyl, ethyl, n-propyl, iso-propyl, n-butyl, sec-butyl, tert-butyl, n-pentyl, neo-pentyl, iso-pentyl, hexyl groups.

The number n in the general formula (I) can be varied depending on the molar ratio of reactants. For example, when the molar ratio of phenolic compound: formaldehyde: amine compound is 1:1:1, the value of n can be 1; if the above molar ratio is 1:2:2, the value of n can be 2.

In the group

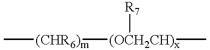

represented by A in the general formula (I), x is varied according to the kind of the amine compound to be used as the starting material. If the amine compound does not include alkylene oxide groups the value of x is zero. If a polyetheramine is used as the amine compound starting material the value of x is the number of repeating ether groups in the polyetheramine. The value of m is also dependent upon the amine groups used.

The phenolic compounds used in the manufacture of the antifoulant of the present invention include, e.g., phenol, methylphenol, dimethylphenol, tert-butylphenol, di-tert-butylphenol, and nonylphenol, but are not limited to these examples. These various phenolic compounds can be used alone or as a combination of two or more.

In the manufacture of the antifoulant of the present invention, the used amine compounds include, e.g., ethylene diamine (EDA), diethylene tri-amine (DETA), triethylene tetramine (TETA), 1,6-hexylenediamine, N,N-dimethyl-1,3-propylenediamine, N,N-dimethyl-1,3-popylenediamine, or polyetheramines of the following formulae

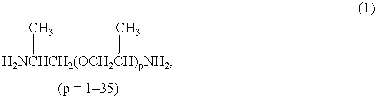

H₂N(CH_{2CH}₂O)₂CH₂CH₂NH_{2tm (}2)

EDR-148

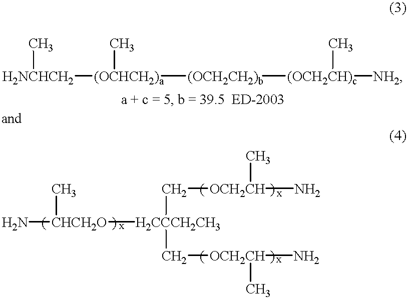

T-403(x=1.5˜2), T-5000 (x=12 ˜13).

In the manufacture of the antifoulant of the present invention the molar ratio of reactants can be widely varied without special limitation, only if it facilitates the production of compounds of antifoulant of the present invention. However, in general the molar ratio of phenolic compounds: formaldehyde: amine compounds can be (1˜5):(1˜5):(1˜5).

The reaction temperature has no special limitation as long as it facilitates the reaction. However, in general, it is within the range of 50 to 200° C., preferrably 80 to 150° C.

The reaction for manufacturing the antifoulant of the present invention is preferably carried out under an inert gas such as nitrogen.

The following examples will be used to explain the present invention in more details. However, the scope of the present invention is not limited to the examples set forth below.

### EXAMPLE 1

Under room temperature, di-tert-butylphenol (12.36 g, 6 mmole) was charged in a three-neck, 250 ml round bottom flask equipped with a thermometer, a temperature controller, a nitrogen inlet and outlet, and a Dean-Stark trap. Under agitation Jaffamine® D-230 (a polyetheramine, p is 2 to 3 in formula(1)) (6.9 g, 3 mmole) was added and mixed well. Then formaldehyde (4.86 g, 6 mmole) was slowly added, heated to 90° C., and reacted for 3 hours. The temperature was then raised to 100˜110° C. and the reaction continued for 1 hour. After removal of most of the water and formaldehyde the temperature was raised to 140° C. and the mixture was allowed to react for 2 hours. Upon completion of the reaction the solvent was removed from the mixture in a rotary evaporator at a temperature of 80° C. and a pressure of 20 mm Hg. A compound of the general formula (I) is obtained, wherein R₁ and R₂ are both tert-butyl groups, n is 1, R₃ is hydrogen, A is a group of

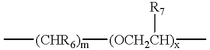

(where R6 is hydrogen, m is 2, x is 2˜3, and R₇ is methyl group), R₄ is hydrogen, and R₅ is a group of

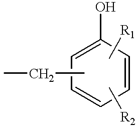

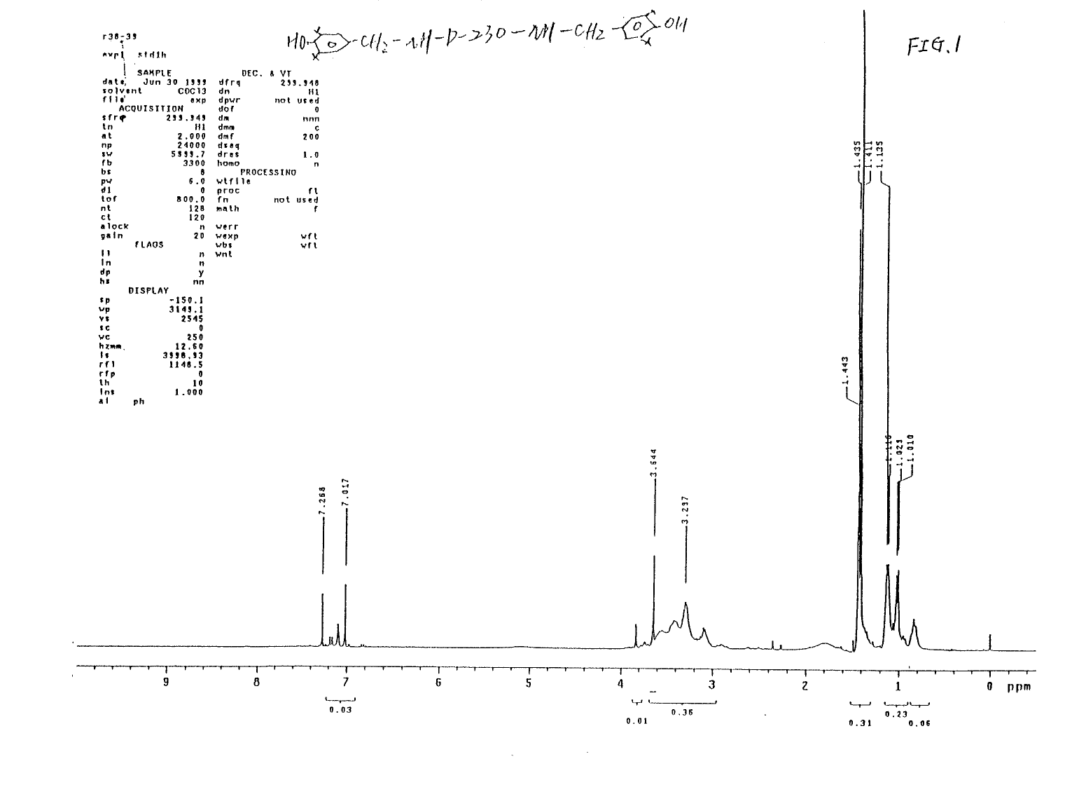

(where R₁ and R₂ are both tert-butyl). FIG. 1 shows the NMR spectrum of this compound. The reaction is also verified by amine titration. The reaction is as follows:

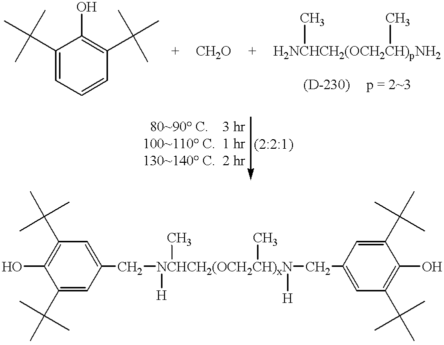

Testing Methods

In accordance with ASTM D3241, a standard testing method of stability of gasoline against oxidation, a jet fuel thermal oxidation tester (Model JFTOPT II, Alcor Petroleum Instruments, Inc. ) was used to carry out tests of antifouling performance. The results are shown in Table 1.

The column of ΔT in Table 1 refers to the temperature difference between the temperature (Tc) at the center of the tester tube through which the heated oil sample flows in and the outlet temperature (Tout) at the end of tube through which the oil flows out. If the antifouling performance is poor a layer of slime deposition on the interior of the tube is formed that reduces heat transfer efficiency thereby ΔT becomes larger. Therefore, when ΔT is smaller, there is less fouling. The value for a crude without antifoulant added is ΔT=15° C., indicating servere fouling. As the antifoulant of the present invention is added ΔT is remarkably reduced, indicating the superior performance by the antifoulant of the present invention.

### EXAMPLES 2-26

The reactants, their quantity, and reaction conditions listed in Table 1 were used in the synthesis of the antifoulants of the present invention in Examples 2 to 26. The testing method of ASTM D3241 is adopted in the tests shown. The results are listed in Table 1 as ΔT.

Data listed in Table 1 provide evidence for the superiority in the performance of the novel antifoulant of the present invention, which can be effectively used in petrochemical plants and oil refineries.

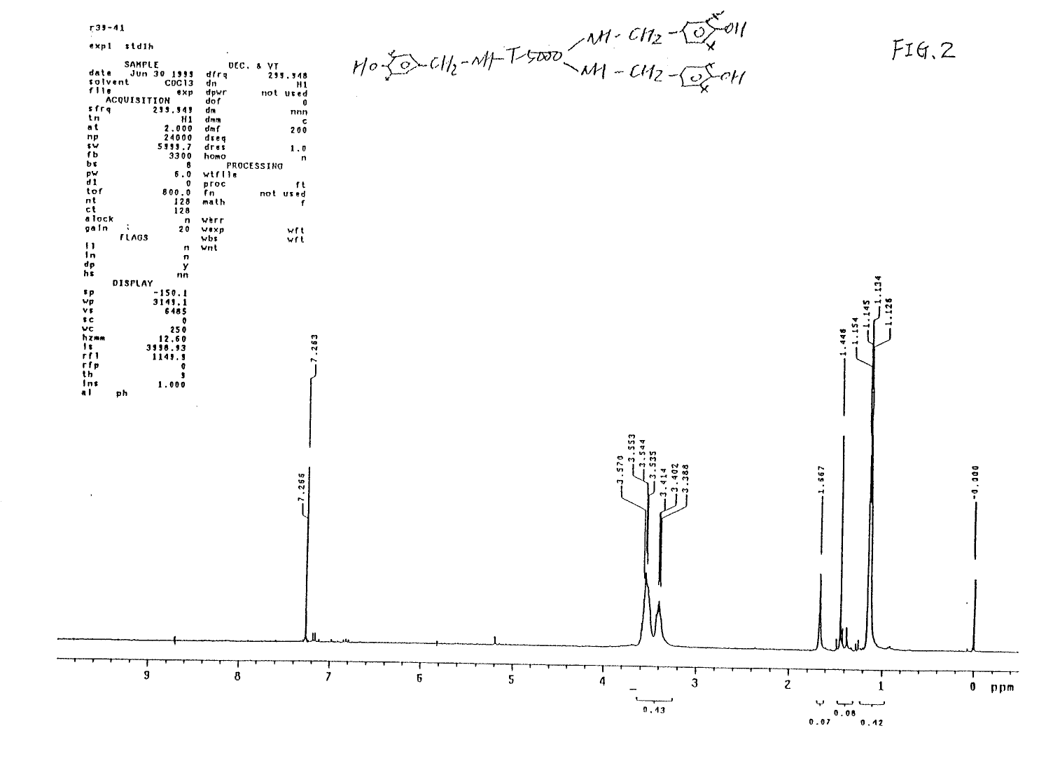

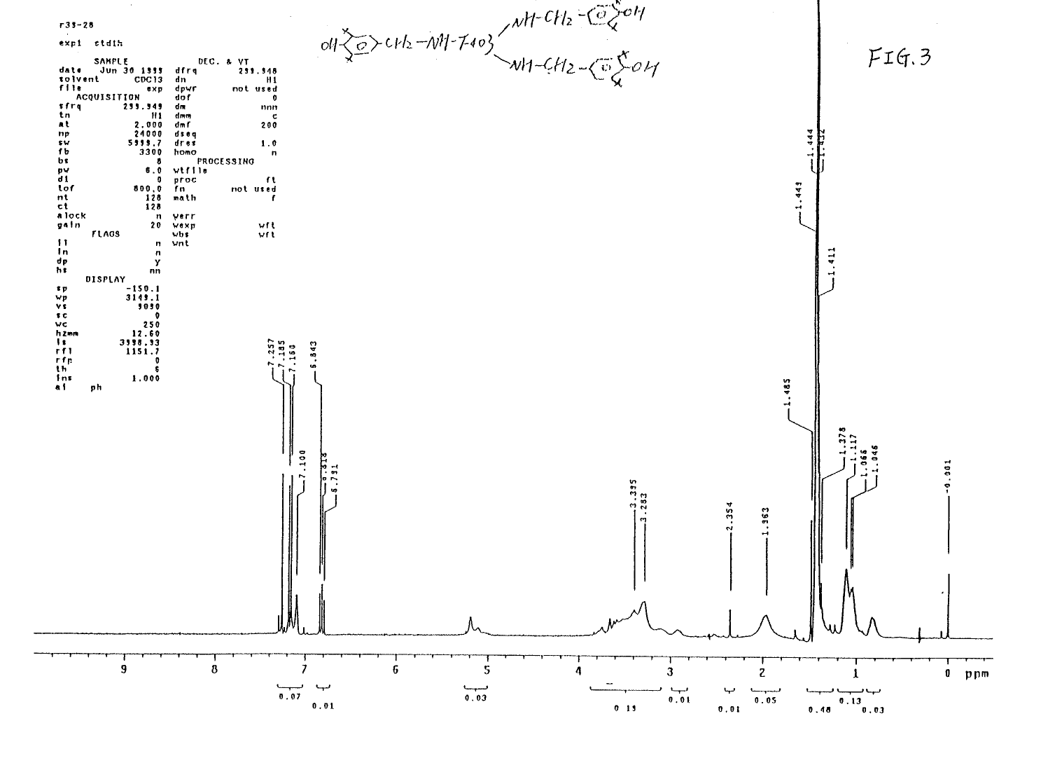

TABLE 1

| | | | | | | Amine | | | | | | | |
|---|---|---|---|---|---|---|---|---|---|---|---|---|---|
| | | | | | | titration | | | | | | | |
| | | | Molar | | Product | theoretical | Total | Primary | Secondary | Tertiary | | | |
| Example | Reactant | Weight (g) | ratio | conditions | recovered | value | amines | amines | amines | amines | Color | ΔT (° C.) | Note |
| 1 | Di-tert- | 12.26 | 2:1:2 | 89-90° C. | 17.23 | 3.14 | 2.6 | 0.3 | 2.3 | 0 | dark green | 9° C. | NMR spectrum |
| | butylphenol | 6.9 | | 3 hr, 130° C. | | | | | | | | | of reaction |
| | (p = 2 ˜ 3 in | 4.89 | | 2 hr | | | | | | | | | product is shown |
| | formula(1)) | | | | | | | | | | | | in FIG. 1 |
| 2 | phenol | 9.77 | 2:1:2 | 89-90° C. | 17.39 | 9.46 | 9.95 | 0.5 | 9.45 | 0 | orange | | |
| | DETA | 10.75 | | 3 hr, 130° C. | | | | | | | | | |
| | CH₂O | 8.43 | | 2 hr | | | | | | | | | |
| 3 | 2−(2- | 11.30 | 1:1:1 | 89-90° C. | 16.33 | 9.48 | 8.61 | 0.50 | 7.57 | 0.99 | yellow | | |
| | aminoethoxy) | 12.89 | | 3 hr, 130° C. | | | | | | | | | |
| | ethanol | 9.75 | | 2 hr | | | | | | | | | |
| 4 | Phenol | 8.28 | 2:1:2 | 89-90° C. | 11.55 | 7.30 | 8.085 | 0.61 | 7.47 | 0 | orange- | | |
| | EDA | 5.45 | | 3 hr, 130° C. | | | | | | | yellow | | |
| | CH₂O | 7.14 | | 2 hr | | | | | | | | | |
| 5 | Phenol | 10.24 | 1:1:1 | 89-90° C. | 14.21 | 11.98 | 8.60 | 1.98 | 6.62 | 0 | orange- | | |
| | EDA | 6.86 | | 3 hr, 130° C. | | | | | | | | yellow | |
| | CH₂O | 9.00 | | 2 hr | | | | | | | | | |
| 6 | Phenol | 6.03 | 1:1:1 | 89-90° C. | 18.72 | 0 | 0 | 0 | 0 | 0 | orange- | | |
| | Phenylene | 11.80 | | 3 hr, 130° C. | | | | | | | black | | |
| | diamine | 5.19 | | 2 hr | | | | | | | | | |
| | CH₂O | | | | | | | | | | | | |
| 7 | N,N- | 4.62 | 1:3:3 | 89-90° C. | 17.6 | 13.76 | 10.97 | 1.92 | 2.83 | 7.22 | pale- | | |
| | Dimethyl- | 150.6 | | 3 hr, 130° C. | | | | | | | yellow | | |
| | 1,3- | 11.97 | | 2 hr | | | | | | | | | |
| | propylenea | | | | | | | | | | | | |
| | mine | | | | | | | | | | | | |
| | CH₂O | | | | | | | | | | | | |
| 8 | Di-tert- | 12.39 | 3:1:3 | 89-90° C. | 19.48 | 2.83 | 2.45 | 0.21 | 2.24 | 0 | orange- | 2° C. | |
| | butylphenol | 8.02 | | 3 hr, 130° C. | | | | | | | green | | |
| | T-403 | 2.50 | | 2 hr | | | | | | | | | |
| | CH₂O | | | | | | | | | | | | |
| 9 | Di-tert- | 10.74 | 2:1:1 | 89-90° C. | 54.07 | 0.82 | 0.80 | 0.30 | 0.50 | 0 | black- | | |
| | butylphenol | 52.14 | | 3 hr, 130° C. | | | | | | | green | | |
| | D-2000 | 2.16 | | 2 hr | | | | | | | | | |
| | (p is about | | | | | | | | | | | | |
| | 33 in formula(1)) | | | | | | | | | | | | |
| | CH₂O | | | | | | | | | | | | |
| 10 | Di-tert- | 3.72 | 3:1:3 | 89-90° C. | 32.53 | 0.58 | 0.40 | 0.32 | 0.06 | 0.02 | dark- | 1° C. | NMR spectrum |
| | butylphenol | 30.06 | | 3 hr, 130° C. | | | | | | | green | | of reaction |
| | T-5000 | 0.55 | | 2 hr | | | | | | | | | product is shown |
| | CH₂O | | | | | | | | | | | | in FIG. 2 |
| 11 | Di-tert- | 10.66 | 1:1:1 | 89-90° C. | 10.85 | 0 | 0 | 0 | 0 | 0 | black- | | |
| | butylphenol | 9.52 | | 3 hr, 130° C. | | | | | | | green | | |
| | CH₂O | 4.19 | | 2 hr | | | | | | | | | |
| 12 | Di-tert- | 5.15 | 1:1:1 | 89-90° C. | 14.2 | 3.24 | 2.61 | 1.49 | 1.12 | 0 | yellow- | | |
| | butylphenol | 10 | | 3 hr, 130° C. | | | | | | | green | | |
| | D-400 | 2.03 | | 2 hr | | | | | | | | | |
| | (p = 5 ˜ 6 in | | | | | | | | | | | | |
| | formula(1)) | | | | | | | | | | | | |
| | CH₂O | | | | | | | | | | | | |
| 13 | Di-tert- | 12.36 | 2:1:2 | 89-90° C. | 21.94 | 2.39 | 1.73 | 0.35 | 1.38 | 0 | green | 6° C. | |
| | butylphenol | 11.98 | | 3 hr, 130° C. | | | | | | | | | |
| | D-400 | 4.86 | | 2 hr | | | | | | | | | |
| | (p = 5 ˜ 6 in | | | | | | | | | | | | |
| | formula(1)) | | | | | | | | | | | | |
| | CH₂O | | | | | | | | | | | | |
| 14 | Di-tert- | 4.13 | 2:1:2 | 89-90° C. | 19.25 | 0.82 | 0.72 | 0.37 | 0.35 | 0 | purple | | |
| | butylphenol | 20 | | 3 hr, 130° C. | | | | | | | | | |
| | ED-2003 | 1.62 | | 2 hr | | | | | | | | | |
| | CH₂O | | | | | | | | | | | | |
| 15 | Di-tert- | 10.03 | 2:1:2 | 89-90° C. | 19.73 | 3.59 | 2.54 | 0.97 | 1.57 | 0 | green | | NMR spectrum |
| | butylphenol | 10 | | 3 hr, 130° C. | | | | | | | | | of reaction |
| | T-403 | 4.06 | | 2 hr | | | | | | | | | product is shown |
| | CH₂O | | | | | | | | | | | | in FIG. 3. |
| 16 | Di-tert- | 5.15 | 1:1:1 | 89-90° C. | 14.55 | 4.85 | 3.72 | 2.22 | 1.50 | 0 | green | | |
| | butylphenol | 10 | | 3 hr, 130° C. | | | | | | | | | |
| | T-403 | 2.03 | | 2 hr | | | | | | | | | |
| | CH₂O | | | | | | | | | | | | |
| 17 | Di-tert- | 10.23 | 1:1:1 | 89-90° C. | 11.56 | 6.25 | 4.88 | 1.02 | 0.76 | 3.1 | tea | 4° C. | |
| | butylphenol | 5.06 | | 3 hr, 130° C. | | | | | | | | | |
| | N,N- | 3.99 | | 2 hr | | | | | | | | | |
| | Dimethyl- | | | | | | | | | | | | |
| | 1,3- | | | | | | | | | | | | |
| | propylenea | | | | | | | | | | | | |
| | mine | | | | | | | | | | | | |
| | CH₂O | | | | | | | | | | | | |
| 18 | Nonyl | 10.05 | 1:1:1 | 89-90° C. | 14.40 | 5.94 | 5.20 | 1.09 | 4.11 | 0 | orange | | |
| | phenol | 1.37 | | 3 hr, 130° C. | | | | | | | | | |
| | 2-(2- | 4.75 | | 2 hr | | | | | | | | | |
| | aminoethoxy | | | | | | | | | | | | |
| | ethanol | | | | | | | | | | | | |
| | CH₂O | | | | | | | | | | | | |
| 19 | Nonyl | 10.00 | 1:1:1 | 89-90° C. | 17.20 | 0 | 0 | 0 | 0 | 0 | black- | | |
| | phenol | 8.36 | | 3 hr, 130° C. | | | | | | | green | | |
| | Phenylene | 3.68 | | 2 hr | | | | | | | | | |
| | diamine | | | | | | | | | | | | |
| | CH₂O | | | | | | | | | | | | |
| 20 | nonyl | 10.00 | 1:1:1 | 89-90° C. | 10.33 | 6.83 | 5.86 | 4.52 | 1.34 | 0 | orange | | |
| | phenol | 2.73 | | 3 hr, 130° C. | | | | | | | transparent | | |
| | EDA | 3.68 | | 2 hr | | | | | | | | | |
| | CH₂O | | | | | | | | | | | | |
| 21 | Nonyl | 22 | 2:1:2 | 89-90° C. | 23.40 | 3.33 | 3.05 | 0.55 | 2.5 | 0 | yellow | | |
| | phenol | 6.81 | | 3 hr, 130° C. | | | | | | | | | |
| | m-dimethylp | 8.11 | | 2 hr | | | | | | | | | |
| | henylene | | | | | | | | | | | | |
| | CH₂O | | | | | | | | | | | | |
| 22 | Nonyl | 12.11 | 3:2:4 | 89-90° C. | 9.44 | 4.08 | 3.63 | 0.09 | 3.54 | 0 | pale | | |
| | phenol | 5 | | 3 hr, 130° C. | | | | | | | yellow | | |
| | m-Dimethylp | 5.95 | | 2 hr | | | | | | | | | |
| | henylene | | | | | | | | | | | | |
| | diamine | | | | | | | | | | | | |
| | CH₂O | | | | | | | | | | | | |
| 23 | Nonyl | 11 | 1:1:1 | 89-90° C. | 13.10 | 5.99 | 4.64 | 0.33 | 1.94 | 2.37 | yellow | 3° C. | |
| | phenol | 5.1 | | 3 hr, 130° C. | | | | | | | | | |
| | N,N- | 4.05 | | 2 hr | | | | | | | | | |
| | dimethyl- | | | | | | | | | | | | |
| | 1,3- | | | | | | | | | | | | |
| | propylened | | | | | | | | | | | | |
| | iamine | | | | | | | | | | | | |
| | CH₂O | | | | | | | | | | | | |
| 24 | Nonyl | 11 | 1:1:1 | 89-90° C. | 14.59 | 2.97 | 2.53 | 0.1 | 2.43 | 0 | yellow | 8° C. | |
| | phenol | 5.25 | | 3 hr, 130° C. | | | | | | | | | |
| | 2-(2- | 4.05 | | 2 hr | | | | | | | | | |
| | aminoethoxy) | | | | | | | | | | | | |
| | ethanol | | | | | | | | | | | | |
| | CH₂O | | | | | | | | | | | | |
| 25 | Nonyl | 11 | 1:2:2 | 89-90° C. | 15.83 | 8.93 | 5.8 | 0.26 | 1.61 | 3.93 | pale | 8° C. | |
| | phenol | 10.2 | | 3 hr, 130° C. | | | | | | | yellow | | |
| | N,N- | 8.1 | | 2 hr | | | | | | | | | |
| | dimethyl- | | | | | | | | | | | | |
| | 1,3- | | | | | | | | | | | | |
| | propylened | | | | | | | | | | | | |
| | iamine | | | | | | | | | | | | |
| | CH₂O | | | | | | | | | | | | |
| 26 | Nonyl | 11 | 1:1:2 | 89-90° C. | 5.8 | | | | | | golden | | |
| | phenol | 6 | | 3 hr, 130° C. | | | | | | | yellow | | |
| | EDA | 8.11 | | 2 hr | | | | | | | | | |
| | CH₂O | | | | | | | | | | | | |

What is claimed is:

1. A compound represented by the following general formula (I), which is useful as an antifoulant, 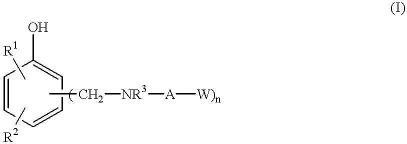
wherein:
R₁ and R₂ are the same or different and represent a C₁₋₁₀ straight chain or branched alkyl group,
A represents a phenylene group which may have a C₁₋₄ alkyl subsitution group, a group of —(CH₂—NR₃—CH₂)_{z}—(where z is an integer of 1˜3) or a group of 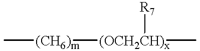
(wherein R₆ and R₇ independently represent a hydrogen atom or straight chain or branched C₁₋₆ alkyl group, m is an integer of 1˜6, and x is an integer of 0˜35),
W represents a hydroxy group or a group of formula-NR₄R₅;
R₃ represents hydrogen atom or C₁₋₆ straight chain or branched -alkyl group,
R4 and R₅ are the same or different and independently represent a hydrogen atom, straight chain or branched C₁₋₆ alkyl group, or a group of the following formula: 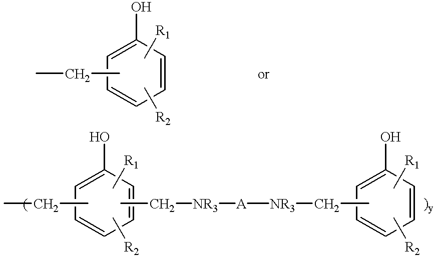
in which R₁, R₂, R₃ and A are as defined above, and y is an integer of 1˜3; and
n represents an integer of 1˜3.

2. The compound of claim 1, wherein the group of formula-(CH₂—NR₃-A-W) is attached to the benzene ring at the meta- and/or para-position with respect tot he hydroxyl group thereof.

3. The compound of claim 1, wherein R₁ and R₂ are independentlya tert-butyl or nonyl group, and R₃ is a hydrogen or a methyl group.

4. The compound in claim 1, which is prepared by the Mannich reaction of phenolic compounds represented by the formula 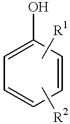
(wherein R₁ and R₂ are as defined in claim 1), formaldehyde, and amine represented by the formula HNR₃-A-W (wherein R_{3,} A and W are as defined in claim 1).

5. The compound of claim 4, wherein the phenolic compounds are selected from the group consisting of phenol, methylphenol, dimethyl-phenol, tert-butylphenol, di-tert-butylphenol, and nonylphenol.

6. The compound of claim 4, wherein the amine compound is selected from the group consisting of ethylenediamine (EDA), diethylenetriamine (DETA), triethylene-tetramine (TETA), 1,6-hexylenediamine, N,N-dimethyl-1,3-propylene-diamine, and polyetheramines represented by the following formula 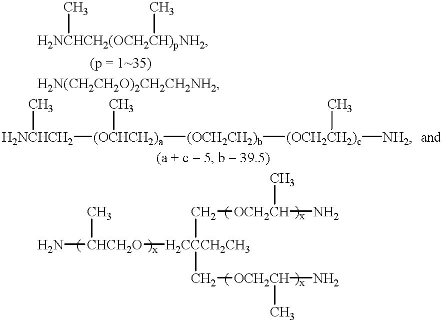
(x is as defined in claim 1).

7. The compound of any one of claims 1 to 6, which is useful as an antifoulant in petrochemical plants or oil refineries.
